(19) United States
(12) Patent Application Publication
(10) Pub. No.: US 2025/0074937 A1
(43) Pub. Date: Mar. 6, 2025

(54) PURIFICATION METHOD OF MYCOBACTERIUM-DERIVED NANOCARRIER PROTEIN

(71) Applicant: Nankai University, Tianjin (CN)

(72) Inventors: Hongri Gong, Tianjin (CN); Zhanqiang Du, Tianjin (CN)

(21) Appl. No.: 18/587,684

(22) Filed: Feb. 26, 2024

(30) Foreign Application Priority Data
Sep. 5, 2023 (CN) 2023111325364

(51) Int. Cl. C07K 1/22; C07K 14/35

(52) U.S. Cl. CPC C07K 1/22; C07K 14/35

(57) ABSTRACT

The present disclosure provides a purification method of a Mycobacterium-derived nanocarrier protein, and belongs to the technical field of protein purification. In the present disclosure, the purification method includes the following steps: providing an Escherichia coli total protein solution, where the Escherichia coli total protein solution includes a Mycobacterium-derived nanocarrier protein, and the Mycobacterium-derived nanocarrier protein is fused with a 1×Flag tag at a C-terminus of a subunit; subjecting the Escherichia coli total protein solution to Flag tag affinity chromatography purification to obtain a crude extract of the Mycobacterium-derived nanocarrier protein; and subjecting the crude extract of the Mycobacterium-derived nanocarrier protein to gel exclusion chromatography purification to obtain a pure product of the Mycobacterium-derived nanocarrier protein. The purification method has simple and easy operations.

### CROSS REFERENCE TO RELATED APPLICATION

This patent application claims the benefit and priority of Chinese Patent Application No. 2023111325364 filed with the China National Intellectual Property Administration on Sep. 5, 2023, the disclosure of which is incorporated by reference herein in its entirety as part of the present application.

### REFERENCE TO SEQUENCE LISTING

A computer readable XML file entitled “GWP20231108560”, that was created on Jan. 10, 2024, with a file size of about 9,630 bytes, contains the sequence listing for this application, has been filed with this application, and is hereby incorporated by reference in its entirety.

### TECHNICAL FIELD

The present disclosure belongs to the technical field of protein purification, and specifically relates to a purification method of a Mycobacterium-derived nanocarrier protein.

### BACKGROUND

Encapsulin is a newly discovered nanocarrier protein that can self-assemble into an regular icosahedral structure. The encapsulin has a diameter of 25 nm to 42 nm, and wraps one or more Cargo proteins internally, thus endowing the encapsulin with multiple physiological functions, such as resistance to oxidative stress. Therefore, in vitro purification to obtain the nanocarrier proteins is of great significance for further transformation and application of the nanocarrier proteins.

Mycobacterium tuberculosis-derived nanocarrier protein was first discovered in the supernatant of a medium, and then named a Culture filtrate protein (CFP) CFP29 due to its constituent subunits size of 29 kDa. Moreover, a sequence of the CFP29 is relatively conserved in different mycobacteria, indicating that Mycobacterium-derived nanocarrier proteins have a similar structure, namely the regular icosahedral structure. Mycobacterium tuberculosis is a pathogenic bacterium that causes tuberculosis. In vitro purification of the Mycobacterium tuberculosis-derived nanocarrier protein as well as other Mycobacterium-derived nanocarrier proteins is of great significance for studying structure function and application of these proteins.

A commonly-used method in this field for the in vitro purification of the Mycobacterium tuberculosis-derived nanocarrier protein as well as other Mycobacterium-derived nanocarrier proteins is generally endogenous extraction, which shows poor yield and purity and is difficult to meet the demands for research.

### SUMMARY

An objective of the present disclosure is to provide a purification method of a Mycobacterium-derived nanocarrier protein. In the present disclosure, the purification method can obtain a high-purity Mycobacterium-derived nanocarrier protein under a low production cost.

The present disclosure provides a purification method of a Mycobacterium-derived nanocarrier protein, including the following steps:
providing an Escherichia coli total protein solution, where the Escherichia coli total protein solution includes a Mycobacterium-derived nanocarrier protein, and the Mycobacterium-derived nanocarrier protein is fused with a 1×Flag tag at a C-terminus of a subunit;
subjecting the Escherichia coli total protein solution to Flagtag affinity chromatography purification to obtain a crude extract of the Mycobacterium-derived nanocarrier protein; and
subjecting the crude extract of the Mycobacterium-derived nanocarrier protein to gel exclusion chromatography purification to obtain a pure product of the Mycobacterium-derived nanocarrier protein.

Preferably, the Mycobacterium-derived nanocarrier protein is one or more selected from the group consisting of a Mycobacterium tuberculosis-derived nanocarrier protein, a Mycobacterium smegmatis-derived nanocarrier protein, a Mycobacterium bovis-derived nanocarrier protein, a Mycobacterium abscessus-derived nanocarrier protein, a Mycobacterium kansasii-derived nanocarrier protein, and a Mycobacterium avium-derived nanocarrier protein.

Preferably, the chromatographic column used for Flag tag affinity chromatography purification is a chromatographic column filled with an Anti-Flag filler; and the chromatographic column used for gel exclusion chromatography purification is a gel exclusion chromatographic column of Superose 6× Increase 10/300.

Preferably, the Flag tag affinity chromatography purification includes first elution and second elution that are conducted sequentially; a first eluent for the first elution includes 2-[4-(2-hydroxyethyl) piperazin-1-yl]ethanesulfonic acid (HEPES), NaCl, and a detergent; and a second eluent for the second elution includes the HEPES, the NaCl, and a 1×Flag peptide.

Preferably, in the first eluent, the HEPES has a molar concentration of 25 mmol/L to 50 mmol/L, the NaCl has a molar concentration of 150 mmol/L to 300 mmol/L, and the detergent has a mass percentage of 0.01% to 0.02%.

Preferably, in the second eluent, the HEPES has a molar concentration of 25 mmol/L to 50 mmol/L, the NaCl has a molar concentration of 150 mmol/L to 300 mmol/L, and the 1×Flag peptide has a mass concentration of 200 μg/mL to 300 μg/mL.

Preferably, the detergent is Tween 20.

Preferably, a third eluent for the gel exclusion chromatography purification includes HEPES and NaCl.

Preferably, in the third eluent, the HEPES has a molar concentration of 25 mmol/L to 50 mmol/L and the NaCl has a molar concentration of 150 mmol/L to 300 mmol/L.

Preferably, a method for preparing the Escherichia coli total protein solution includes the following steps:
cloning a fusion gene of the Mycobacterium-derived nanocarrier protein into a pETDuet-1 plasmid to obtain a recombinant plasmid;
transferring the recombinant plasmid into Escherichia coli to allow prokaryotic expression to obtain an Escherichia coli engineered strain; and
resuspending the Escherichia coli engineered strain with a resuspension reagent, and collecting a resulting supernatant after solid-liquid separation to obtain the Escherichia coli total protein solution.

The present disclosure provides a purification method of a Mycobacterium-derived nanocarrier protein, including the following steps: providing an Escherichia coli total protein solution, where the Escherichia coli total protein solution includes a Mycobacterium-derived nanocarrier protein, and the Mycobacterium-derived nanocarrier protein is fused with a 1×Flag tag at a C-terminus of a subunit; subjecting the Escherichia coli total protein solution to Flag tag affinity chromatography purification to obtain a crude extract of the Mycobacterium-derived nanocarrier protein; and subjecting the crude extract of the Mycobacterium-derived nanocarrier protein to gel exclusion chromatography purification to obtain a pure product of the Mycobacterium-derived nanocarrier protein. In the present disclosure, the purification method utilizes the 1×Flag tag at the C-terminus of the subunit of the Mycobacterium-derived nanocarrier protein. The Flag tag is used for affinity chromatography purification, and the Anti-Flag filler is used for specifically identification of the 1×Flag tag. Therefore, a crude extract rich in the Mycobacterium-derived nanocarrier protein can be effectively obtained, and then a purified Mycobacterium-derived nanocarrier protein can be obtained through gel exclusion chromatography. The purification method has simple operation process and a low cost. The obtained Mycobacterium-derived nanocarrier protein has a higher purity and a larger yield, and can be used for subsequent researches on structure function and drug delivery of the Mycobacterium-derived nanocarrier protein.

### BRIEF DESCRIPTION OF THE DRAWINGS

To describe the technical solutions in examples of the present disclosure or in the prior art more clearly, the drawings required in the examples are briefly described below. Apparently, the drawings in the following description show merely some examples of the present disclosure, and other drawings can be derived from these drawings by those of ordinary skill in the art without creative efforts.

FIG. 1 shows results of gel exclusion chromatography purification in Example 1; where in FIG. 1, an abscissa represents an elution volume of the second eluent in a unit of mL; and a left ordinate represents an absorbance of a UV spectrophotometer at 280 nm in a unit of mAU;

FIG. 2 shows an SDS-PAGE electrophoresis diagram of the gel exclusion chromatography purification in Example 1; where in FIG. 2, the left represents a Marker and the right represents sample identification;

FIG. 3 shows a negative staining electron microscope image of the Mycobacterium tuberculosis-derived nanocarrier protein in Example 1 after gel exclusion chromatography purification; and

FIG. 4 shows a structure of the Mycobacterium tuberculosis-derived nanocarrier protein in Example 1.

### DETAILED DESCRIPTION OF THE EMBODIMENTS

The present disclosure provides a purification method of a Mycobacterium-derived nanocarrier protein, including the following steps:

providing an Escherichia coli total protein solution, where the Escherichia coli total protein solution includes a Mycobacterium-derived nanocarrier protein, and the Mycobacterium-derived nanocarrier protein is fused with a 1×Flag tag at a C-terminus of a subunit;
subjecting the Escherichia coli total protein solution to Flag tag affinity chromatography purification to obtain a crude extract of the Mycobacterium-derived nanocarrier protein; and
subjecting the crude extract of the Mycobacterium-derived nanocarrier protein to gel exclusion chromatography purification to obtain a pure product of the Mycobacterium-derived nanocarrier protein.

In the present disclosure, unless otherwise specified, the raw materials used are all commercially-available products well known to those skilled in the art.

In the present disclosure, an Escherichia coli total protein solution is provided, where the Escherichia coli total protein solution includes a Mycobacterium-derived nanocarrier protein, and the Mycobacterium-derived nanocarrier protein is fused with a 1×Flag tag at a C-terminus of a subunit. In the present disclosure, the Mycobacterium-derived nanocarrier protein is preferably one or more selected from the group consisting of a Mycobacterium tuberculosis-derived nanocarrier protein, a Mycobacterium smegmatis-derived nanocarrier protein, a Mycobacterium bovis-derived nanocarrier protein, a Mycobacterium abscessus-derived nanocarrier protein, a Mycobacterium kansasii-derived nanocarrier protein, and a Mycobacterium avium-derived nanocarrier protein, more preferably includes the Mycobacterium tuberculosis-derived nanocarrier protein, the Mycobacterium smegmatis-derived nanocarrier protein, the Mycobacterium bovis-derived nanocarrier protein, the Mycobacterium abscessus-derived nanocarrier protein, the Mycobacterium kansasii-derived nanocarrier protein, and the Mycobacterium avium-derived nanocarrier protein.

In the present disclosure, a method for preparing the Escherichia coli total protein solution includes the following steps: cloning a fusion gene of the Mycobacterium-derived nanocarrier protein into a pETDuet-1 plasmid to obtain a recombinant plasmid; transferring the recombinant plasmid into Escherichia coli to allow prokaryotic expression to obtain an Escherichia coli engineered strain; and resuspending the Escherichia coli engineered strain with a resuspension reagent, and collecting a resulting supernatant after solid-liquid separation to obtain the Escherichia coli total protein solution. The 1×Flag tag is preferably fused to the C-terminus of the subunit of the Mycobacterium-derived nanocarrier protein by molecular cloning methods. There are no special requirements for the molecular cloning methods, and the molecular cloning methods well known to those skilled in the art can be used. In the present disclosure, the resuspension reagent preferably includes HEPES, NaCl, and a detergent; in the resuspension reagent, the HEPES has a molar concentration of preferably 25 mmol/L to 50 mmol/L, more preferably 25 mmol/L; the NaCl has a molar concentration of preferably 150 mmol/L to 300 mmol/L, more preferably 150 mmol/L; and the detergent has a mass percentage of preferably 0.01% to 0.02%, more preferably 0.01%. The detergent is preferably Tween 20. The method for resuspending is preferably high-pressure crushing. There are no special requirements for a specific operation of the high-pressure crushing, and the high-pressure crushing operation methods familiar to those skilled in the art can be used. In the present disclosure, the solid-liquid separation is preferably conducted by centrifugation. There are no special requirements for specific conditions of the centrifugation, and centrifugation conditions well known to those skilled in the art can be used.

In the present disclosure, the Escherichia coli total protein solution is obtained and then subjected to Flag tag affinity chromatography purification to obtain a crude extract of the Mycobacterium-derived nanocarrier protein. In the present disclosure, the Flag tag affinity chromatography purification is preferably conducted on a chromatographic column filled with an Anti-Flag filler. In the present disclosure, the Flag tag affinity chromatography purification preferably includes first elution and second elution that are conducted sequentially. A first eluent for the first elution preferably includes HEPES, NaCl, and a detergent; in the first eluent, the HEPES has a molar concentration of preferably 25 mmol/L to 50 mmol/L, more preferably 25 mmol/L; the NaCl has a molar concentration of preferably 150 mmol/L to 300 mmol/L, more preferably 150 mmol/L; the detergent has a mass percentage of preferably 0.01% to 0.02%, more preferably 0.01%; and the detergent is preferably Tween 20. In the present disclosure, the first eluent has a pH value of preferably 7.4. In the present disclosure, the first eluent has a volume preferably 5 to 10 times that of the chromatographic column filled with the Anti-Flag filler. In the present disclosure, function of the first elution is to remove impurities.

In the present disclosure, the second elution is preferably competitive elution; a second eluent for the second elution preferably includes HEPES, NaCl, and a 1×Flag peptide; in the second eluent, the HEPES has a molar concentration of preferably 25 mmol/L to 50 mmol/L, more preferably 25 mmol/L; the NaCl has a molar concentration of preferably 150 mmol/L to 300 mmol/L, more preferably 150 mmol/L; and the 1×Flag peptide has a mass concentration of preferably 200 μg/mL to 300 μg/mL, more preferably 200 μg/mL. The second eluent has a pH value of preferably 7.4. The second eluent has a volume preferably 5 to 10 times that of the chromatographic column filled with the Anti-Flag filler. The 1×Flag peptide has an amino acid sequence set forth in SEQ ID NO: 1; and the SEQ ID NO: 1 is: DYKDDDDK. In the present disclosure, function of the second elution is to obtain the crude extract of the Mycobacterium-derived nanocarrier protein.

In the present disclosure, after the Flag tag affinity chromatography purification is completed, an eluate obtained from the second elution is subjected to solid-liquid separation and concentration to obtain the crude extract of the Mycobacterium-derived nanocarrier protein. In the present disclosure, the eluate is preferably subjected to the solid-liquid separation by centrifugation. There are no special requirements for specific conditions of the centrifugation, and centrifugation conditions well known to those skilled in the art can be used. In the present disclosure, the concentration is preferably centrifugal concentration, and the centrifugal concentration is preferably conducted in a concentration tube with a molecular weight cut-off of 100 kDa. There are no special requirements in the present disclosure for a specific operation of the centrifugal concentration, as long as it can meet the subsequent gel exclusion chromatography purification. In an example of present disclosure, the eluate obtained from the second elution is concentrated by centrifugation to not more than 1,000 μL.

In the present disclosure, the crude extract of the Mycobacterium-derived nanocarrier protein is subjected to gel exclusion chromatography purification to obtain a pure product of the Mycobacterium-derived nanocarrier protein. In the present disclosure, the gel exclusion chromatography purification is preferably conducted on a gel exclusion chromatographic column of Superose 6× Increase 10/300. In the present disclosure, the gel exclusion chromatography purification preferably includes third elution. A third eluent for the gel exclusion chromatography purification in the present disclosure preferably includes HEPES and NaCl; where the HEPES has a molar concentration of preferably 25 mmol/L to 50 mmol/L, more preferably 25 mmol/L and the NaCl has a molar concentration of preferably 150 mmol/L to 300 mmol/L, more preferably 150 mmol/L. In the present disclosure, the third eluent has a pH value of preferably 7.4. In the present disclosure, the third eluent has a volume of preferably 25 mL. In the present disclosure, the third elution is intended to obtain the pure product of the Mycobacterium-derived nanocarrier protein.

In the present disclosure, a recombinant plasmid constructed with a pETDuet-1 vector is transferred to Escherichia coli to obtain an experimental material, and the Flag tag affinity chromatography purification and gel exclusion chromatography purification are adopted in combination. This process has simple steps, short purification cycle, and low requirements for experimental instruments and equipment, thereby greatly reducing time and capital costs.

The technical solutions of the present disclosure will be clearly and completely described below with reference to the example of the present disclosure. Apparently, the described example are merely a part rather than all of the examples of the present disclosure. All other examples obtained by those skilled in the art based on the examples of the present disclosure without creative efforts shall fall within the protection scope of the present disclosure.

In the example of the present disclosure, the chromatographic column for the Flag tag affinity chromatography purification is a commercial product, with product information of Anti-DYKDDDDK G1 Affinity Resin (Genscript Biotechnology Co., Ltd.); and the chromatographic column for the gel exclusion chromatography purification is a commercial product, with product information of Superose 6× Increase 10/300 (GE Healthcare);
the Tween 20 has product information of Tween 20 (Biofroxx); and
all other reagents used in the example are commercial products.

### Example 1

A first eluent, a second eluent, and a third eluent were prepared using ddH₂O as a solvent:
the first eluent (pH=7.4) included: 25 mmol/L of HEPES, 150 mmol/L of NaCl, and 0.02% of Tween 20 by mass percentage;
the second eluent (pH=7.4) included: 25 mmol/L of HEPES, 150 mmol/L of NaCl, and 200 μg/mL of 1× Flag peptide; and
the third eluent (pH=7.4) included: 25 mmol/L of HEPES and 150 mmol/L of NaCl.

A fusion gene of the Mycobacterium-derived nanocarrier protein was cloned into a pETDuet-1 plasmid using molecular cloning to obtain a recombinant plasmid; the recombinant plasmid was transferred into Escherichia coli to allow prokaryotic expression to obtain an Escherichia coli engineered strain; the Escherichia coli engineered strain was subjected to high-pressure crushing using the first eluent, centrifuged, and a resulting supernatant was collected to obtain an Escherichia coli total protein solution; where a C-terminus of a subunit of the Mycobacterium-derived nanocarrier protein in the Escherichia coli total protein solution was fused with a 1×Flag tag.

2 L of the Escherichia coli total protein solution was loaded onto a chromatographic column filled with an Anti-Flag filler to allow Flag tag affinity chromatography purification, specifically including: first elution and second elution were conducted sequentially with 50 mL of the first eluent and 50 mL of the second eluent, respectively, an eluate obtained from the second elution was collected and placed in a concentration tube with a molecular weight cut-off of 100 kDa, and subjected to centrifugal concentration to not more than 1,000 μL to obtain a crude extract of the Mycobacterium-derived nanocarrier protein.

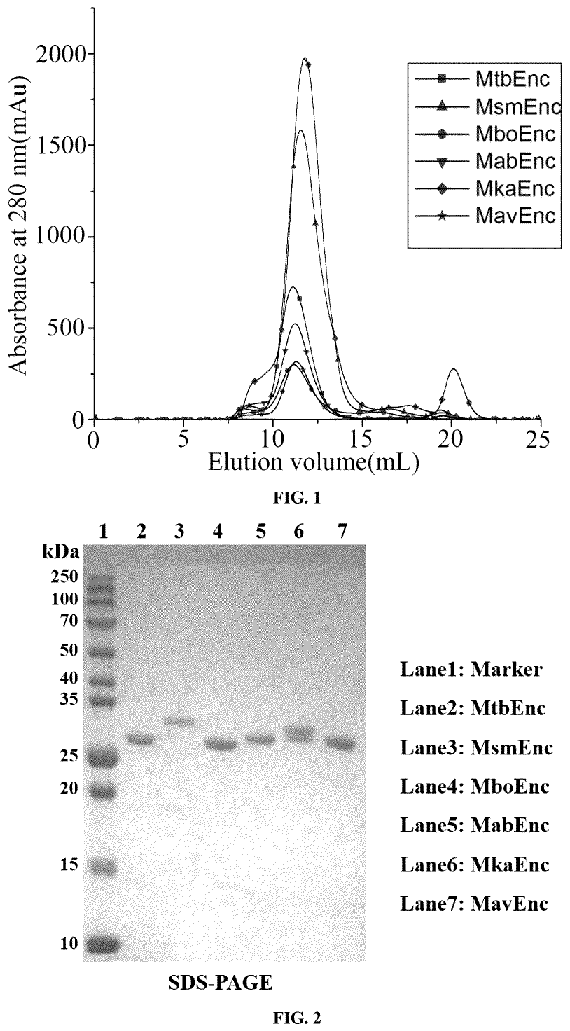

A gel exclusion chromatographic column of Superose 6× Increase 10/300 was equilibrated with 1 column volume (25 mL) of the third eluent, then the crude extract of the Mycobacterium-derived nanocarrier protein was loaded on the gel exclusion chromatographic column of Superose 6× Increase 10/300, and eluted with 25 mL of the third eluent. The results were shown in FIG. 1. In FIG. 1, an abscissa was an elution volume of the chromatographic column in a unit of mL; while a left ordinate was an absorbance of the UV spectrophotometer at 280 nm in a unit of mAU. As shown in FIG. 1, the Mycobacterium-derived nanocarrier protein showed characteristic peaks at 10 mL to 12.5 mL, where the Mycobacterium-derived nanocarrier protein included a Mycobacterium tuberculosis-derived nanocarrier protein (MtbEnc, with an amino acid sequence set forth in SEQ ID NO:2), a Mycobacterium smegmatis-derived nanocarrier protein (MsmEnc, with an amino acid sequence set forth in SEQ ID NO:3), a Mycobacterium bovis-derived nanocarrier protein (MboEnc, with an amino acid sequence set forth in SEQ ID NO:4), a Mycobacterium abscessus-derived nanocarrier protein (MabEnc, with an amino acid sequence set forth in SEQ ID NO:5), a Mycobacterium kansasii-derived nanocarrier protein (MkaEnc, with an amino acid sequence set forth in SEQ ID NO:6), and a Mycobacterium avium-derived nanocarrier protein (MavEnc, with an amino acid sequence set forth in SEQ ID NO:7). The sequences of different Mycobacterium-derived nanocarrier proteins were relatively conserved.

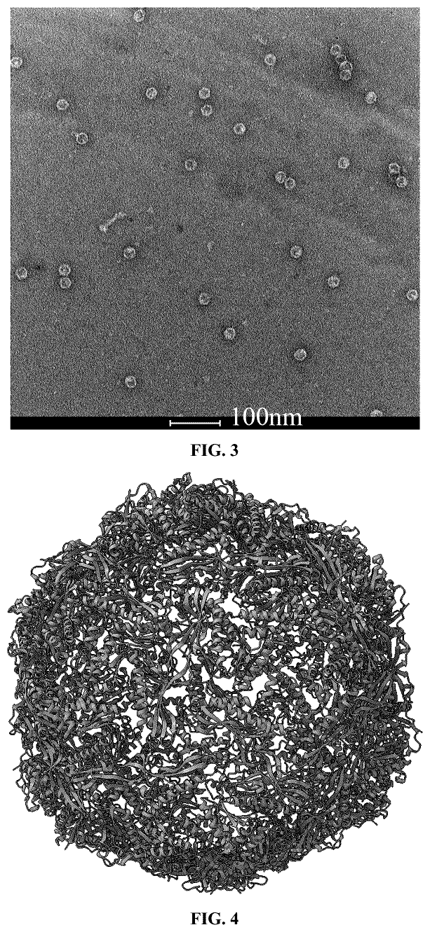

The samples at characteristic peak positions were collected to obtain a Mycobacterium-derived nanocarrier protein solution. The collected Mycobacterium-derived nanocarrier protein solution was subjected to SDS-PAGE electrophoresis, and the detection results were shown in FIG. 2. According to FIG. 2, it was determined that the samples at characteristic peak positions were the Mycobacterium-derived nanocarrier protein. Negative staining electron microscopy was conducted on the Mycobacterium tuberculosis-derived nanocarrier protein sample, and the results were shown in FIG. 3. It was seen from FIG. 3 that the sample had a uniform particle size. The structure of Mycobacterium tuberculosis-derived nanocarrier protein (PDB: 8IKA) was shown in FIG. 4. A molecule of the Mycobacterium tuberculosis-derived nanocarrier protein was composed of 60 subunits and had a regular icosahedral structure.

In the present disclosure, the purification method has simple operations, lower cost, and shorter extraction time compared with those of the endogenous extraction. The obtained Mycobacterium-derived nanocarrier protein shows high purity and large yield, and can be used for subsequent further researches on structure function and drug delivery of the Mycobacterium-derived nanocarrier protein.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A purification method of a Mycobacterium-derived nanocarrier protein, comprising the following steps:
providing an Escherichia coli total protein solution, wherein the Escherichia coli total protein solution comprises a Mycobacterium-derived nanocarrier protein, and the Mycobacterium-derived nanocarrier protein is fused with a 1×Flag tag at a C-terminus of a subunit;
subjecting the Escherichia coli total protein solution to Flag tag affinity chromatography purification to obtain a crude extract of the Mycobacterium-derived nanocarrier protein; and
subjecting the crude extract of the Mycobacterium-derived nanocarrier protein to gel exclusion chromatography purification to obtain a pure product of the Mycobacterium-derived nanocarrier protein.

2. The purification method according to claim 1, wherein the Mycobacterium-derived nanocarrier protein is one or more selected from the group consisting of a Mycobacterium tuberculosis-derived nanocarrier protein, a Mycobacterium smegmatis-derived nanocarrier protein, a Mycobacterium bovis-derived nanocarrier protein, a Mycobacterium abscessus-derived nanocarrier protein, a Mycobacterium kansasii-derived nanocarrier protein, and a Mycobacterium avium-derived nanocarrier protein.

3. The purification method according to claim 1, wherein a chromatographic column used for the Flag tag affinity chromatography purification is a chromatographic column filled with an Anti-Flag filler; and a chromatographic column used for the gel exclusion chromatography purification is a gel exclusion chromatographic column of Superose 6% Increase 10/300.

4. The purification method according to claim 1, wherein the Flag tag affinity chromatography purification comprises first elution and second elution that are conducted sequentially; a first eluent for the first elution comprises 2-[4-(2-hydroxyethyl) piperazin-1-yl]ethanesulfonic acid (HEPES), NaCl, and a detergent; and a second eluent for the second elution comprises the HEPES, the NaCl, and a 1×Flag peptide.

5. The purification method according to claim 4, wherein in the first eluent, the HEPES has a molar concentration of 25 mmol/L to 50 mmol/L, the NaCl has a molar concentration of 150 mmol/L to 300 mmol/L, and the detergent has a mass percentage of 0.01% to 0.02%.

6. The purification method according to claim 4, wherein in the second eluent, the HEPES has a molar concentration of 25 mmol/L to 50 mmol/L, the NaCl has a molar concentration of 150 mmol/L to 300 mmol/L, and the 1×Flag peptide has a mass concentration of 200 μg/mL to 300 μg/mL.

7. The purification method according to claim 4, wherein the detergent is Tween 20.

8. The purification method according to claim 1, wherein a third eluent for the gel exclusion chromatography purification comprises HEPES and NaCl.

9. The purification method according to claim 8, wherein in the third eluent, the HEPES has a molar concentration of 25 mmol/L to 50 mmol/L and the NaCl has a molar concentration of 150 mmol/L to 300 mmol/L.

10. The purification method according to claim 1, wherein a method for preparing the Escherichia coli total protein solution comprises the following steps:
cloning a fusion gene of the Mycobacterium-derived nanocarrier protein into a pETDuet-1 plasmid to obtain a recombinant plasmid;
transferring the recombinant plasmid into Escherichia coli to allow prokaryotic expression to obtain an Escherichia coli engineered strain; and
resuspending the Escherichia coli engineered strain with a resuspension reagent, and collecting a resulting supernatant after solid-liquid separation to obtain the Escherichia coli total protein solution.

11. The purification method according to claim 4, wherein a chromatographic column used for the Flag tag affinity chromatography purification is a chromatographic column filled with an Anti-Flag filler, and a chromatographic column used for the gel exclusion chromatography purification is a gel exclusion chromatographic column of Superose 6® Increase 10/300.


SEQUENCE LISTING (7 sequences)

SEQ ID NO: 1 - protein, 8 aa, synthetic construct
  source 1..8: note = Peptide
DYKDDDDK

SEQ ID NO: 2 - protein, 265 aa, synthetic construct
  source 1..265: note = MtbEnc
MNNLYRDLAPVTEAAWAEIELEAARTFKRHIAGRRVVDVSDPGGPVTAAVSTGRLIDVKAPTNGVIAHLRASKPLVRLRVPFTLSRNEIDDVERGSKDSDWEPVKEAAKKLAFVEDRTIFEGYSAASIEGIRSASSNPALTLPEDPREIPDVISQALSELRLAGVDGPYSVLLSADVYTKVSETSDHGYPIREHLNRLVDGDIIWAPAIDGAFVLTTRGGDFDLQLGTDVAIGYASHDTDTVRLYLQETLTFLCYTAEASVALSH

SEQ ID NO: 3 - protein, 265 aa, synthetic construct
  source 1..265: note = MsmEnc
MNNLYRDLAPITESAWAEIELEATRTFKRHIAGRRVVDVSGPNGPTTASVSTGHLLDVSPPGDGVIAHLRDAKPLVRLRVPFTVARRDIDDVERGSQDSDWDPVKDAAKKLAFVEDRAIFEGYAAASIEGIRSSSSNPALALPDDAREIPDVIAQALSELRLAGVDGPYSVLLSAETYTKVSETTAHGYPIREHINRLVDGEIIWAPAIDGAFVLSTRGGDFDLQLGTDVSIGYLSHDAEVVHLYMEETMTFLCYTAEASVALTP

SEQ ID NO: 4 - protein, 265 aa, synthetic construct; identical to SEQ ID NO: 2
  source 1..265: note = MboEnc

SEQ ID NO: 5 - protein, 265 aa, synthetic construct
  source 1..265: note = MabEnc
MNNLYRELAPITDEAWAEIENEAARTFKRHIAGRRVVDVSEPGGPSSAAVSTGHLLDVTAPADGVQAHLRESKPLVRLRVPFTISRSAIDDVERGAQDSDWDPVKEAAKKLAFAEDRAIFEGYPAASIVGIRESSSNPELKLPEDVREYPDIVAQALSELRLAGVDGPYSVLLSAEEYTKVSEASDRGYPIREHLRRLVTGEILWAPAIDGAFVLTCRGGDFDLQLGTDVTIGYLSHDAGSVQLYLQETLTFLSYTAEASVALLP

SEQ ID NO: 6 - protein, 268 aa, synthetic construct
  source 1..268: note = MkaEnc
MNNLYRELAPVTEAAWAEIELEATRTFKRHIAGRRVVDVSGPGGPVAAAVSTGRLIDISAPTDGVIAHLRASKPLVRLRVPFALSRNEIDDVERGSQDSDWDPVKAAAKKLAFVEDRTIFEGYAAASIEGIRSASSNRPLTLPEDPREIPDVISQALTELRLAGVDGPYSVLLAADVYTKVSETTEHGYPIREHLNRLVDGDIIWAPAIDGAFVLTTRGGDFDLQLGTDVAIGYTSHDADTVQLYLQETLTFLCYTAEASVALSPPSD

SEQ ID NO: 7 - protein, 265 aa, synthetic construct
  source 1..265: note = MavEnc
MNNLYRDLAPVTEAAWGEIELEASRTFKRHVAGRRVVDVSEPGGPAAAAVSTGRLIDVEAPTNGVVAHLRASKPLVRLRVPFTLSRYEIDNVERGANDSDWDPVKEAAKKLAFVEDRAIFEGYAAASIDGIRSASSNKPLALPTDPREIPDVITQAISELRLAGVDGPYSVLLSADVYTKVSETTEHGYPILEHIDRLVPGDIIWAPAIDGAFVLTTRGGDFDLQLGTDVSIGYTSHDADTVQLYLQETLTFLCYTAEAAVPLTS